(19) United States
(12) Patent Application Publication
(10) Pub. No.: US 2025/0331479 A1
(43) Pub. Date: Oct. 30, 2025

(54) Anthocyanin-Free Broccoli

(71) Applicant: Bejo Zaden B.V., Warmenhuizen (NL)

(72) Inventors: Johannes Theodorus Wilhelmus Ligthart, Warmenhuizen (NL); Jan Sybe Wijngaarden, Warmenhuizen (NL); Marcel Adriaanse, Warmenhuizen (NL); Roelof Marinus Veenstra, Warmenhuizen (NL); Albertus Johannes Maria Schrijver, Warmenhuizen (NL)

(21) Appl. No.: 18/869,959

(22) PCT Filed: May 11, 2023
(86) PCT No.: PCT/EP2023/062639

§ 371 Date: Nov. 27, 2024

(30) Foreign Application Priority Data
May 31, 2022 (NL) 2032030

(51) Int. Cl. A01H 1/00; A01H 1/04; C12N 15/82

(52) U.S. Cl. CPC A01H 1/107; A01H 1/04; C12N 15/825

(57) ABSTRACT

Provided herein is an anthocyanin-free broccoli plant including a combination of an inactive MYB2 gene and an inactive DFR1 gene. The present invention further relates to methods for providing the present anthocyanin-free broccoli plants. The present invention also relates to seeds and plant parts of the present anthocyanin-free broccoli plants.

### CROSS-REFERENCE TO RELATED APPLICATIONS

This application is the United States national phase of International Patent Application No. PCT/EP2023/062639 filed May 11, 2023, and claims priority to The Netherlands Patent Application No. 2032030 filed May 31, 2022, the disclosures of which are hereby incorporated by reference in their entireties.

### SEQUENCE LISTING

The Sequence Listing associated with this application is filed in electronic format via Patent Center and is hereby incorporated by reference into the specification in its entirety. The name of the file containing the Sequence Listing is 2407799.xml. The size of the file is 9,111 bytes, and the file was created on Nov. 19, 2024.

### BACKGROUND OF THE INVENTION

### Field of the Invention

The present invention relates to an anthocyanin-free broccoli plant comprising a combination of an inactive MYB2 gene and an inactive DFR1 gene. The present invention further relates to methods for providing the present anthocyanin-free broccoli plants. The present invention also relates to seeds and plant parts of the present anthocyanin-free broccoli plants.

Broccoli is a well-known vegetable in the plant species Brassica oleracea. It is grown for its large flowering head, which in its immature form is eaten as a vegetable.

The plant species B. oleracea includes many common vegetables, such as cauliflower, kale and cabbage. Vegetables in the species Brassica oleracea can be divided into eight cultivars depending on their phenotypic traits: Brassica oleracea var. acephala (kale and collard greens); Brassica oleracea var. alboglabra, (Chinese kale); Brassica oleracea var. botrytris (cauliflower and Romanesco broccoli); Brassica oleracea var. capitata (cabbage and Savoy cabbage); Brassica oleracea var. gemmifera (Brussels sprouts); Brassica oleracea var. gongylodes (kohlrabi); Brassica oleracea var. italica (broccoli); and Brassica oleracea var. sabellica (curly and Portuguese kale). All of these cultivars are derived from wild cabbage that is native to Southern and Western Europe by selection for a specific part of the plant. Kale and cabbage have been selected for their prominent leaves, Brussels sprouts for their axial buds, cauliflower for its white inflorescence meristem, and broccoli for its large flower head.

Broccoli is a cool-season crop, which generally prefers temperatures between 18 and 23°C. Depending on the variety and climate conditions, broccoli requires about 50 to 115 days from seed to harvest. Broccoli is harvested when the flower head is fully developed, but before the individual green buds open and display small yellow flowers. Although the flower head of broccoli is generally green, it is known that the flower head can turn a little purple under cold conditions. This purple discolouration is caused by the production of anthocyanin.

Anthocyanins are a large group of secondary metabolites that belong to the group of flavonoids. Depending on the pH of their environment, they can be orange, red, purple, or blue in colour. Anthocyanins are widely found in higher plants and can give colour to various plant parts, including leaves, roots, stems, flowers, fruits, and seeds. Anthocyanins perform diverse functions in plants, such as attracting pollinators like insects and protecting the plant against exposure to UV, cold temperatures, and drought stresses.

### Description of Related Art

Biosynthesis, regulation, and transport of anthocyanins have been well studied, especially in the model plant Arabidopsis thaliana. The anthocyanin biosynthetic pathway begins with the conversion of phenylalanine to p-coumaroyl-CoA by the phenylpropanoid pathway genes phenylalanine ammonia-lyase (PAL), cinnamate 4-hydroxylase (C4H), and 4-coumarate-CoA ligase (4CL). Subsequently p-coumaroyl-CoA is condensed with malonyl-CoA and converted into flavonols by the early biosynthetic enzymes chalcone synthase (CHS), chalcone isomerase (CHI), flavanone 3-hydroxylase (F3H) and flavonoid 3′-hydroxylase (F3′H). These flavonols are then oxidized by flavonol synthase (FLS) or used as a substrate by flavonoid 3′-hydroxylase (F3′H), flavonoid 3′,5′-hydroxylases (F3′5′H), dihydroflavonol 4-reductase (DFR) and anthocyanidin synthase (ANS), also known as leucoanthocyanidin dioxygenase (LDOX), to produce various anthocyanidins. Anthocyanidins can undergo further modifications, such as glycosylation, acylation, methylation or a combination thereof. Anthocyanidins in plants mainly exist in glycosidic forms commonly referred to as anthocyanins. Glycosolyation of anthocyanidins is catalysed by the enzyme UDP-glycosyltransferase (UGT). After synthesis in the cytosol, anthocyanins are transported into vacuoles via glutathione S-transferases (GSTs), the ATP-binding cassette (ABC), and multidrug and toxic compound extrusion (MATE) proteins.

The biosynthetic genes are regulated primarily at the transcriptional level, especially by R2R3-MYB protein family members. In Arabidopsis and other eudicots, the phenylpropanoid pathway and early anthocyanin biosynthetic genes are regulated by R2R3-MYB proteins, such as MYB11, MYB12, and MYB111. Late anthocyanin biosynthetic genes, such as DRF and ANS, are activated by the MYB-bHLH-WDR (MBW) transcriptional activation complex, which comprises one R2R3-MYB protein (AtMYB75/PAP1, AtMYB90/PAP2, AtMYB112, MYB113, or MYB114), one bHLH protein (GL3, EGL3, or TT8), and a protein called Transparent Testa Glabrous1 (TTG1, also known as WD40). The R2R3-MYB transcription factors, like AtMYB75/PAP1, AtMYB90/PAP2, AtMYB112, AtMYB113, and AtMYB114, are positive regulators that initiate and activate the MBW complex. In addition, two single-repeat R3-MYB transcription factors, MYBL2 and CAPRICE (CPC), and three Lateral Organ Boundary Domain (LBD) gene family members called LBD37, LBD38, and LBD39, negatively regulate anthocyanin biosynthesis in Arabidopsis. The various transcriptional modulators control, besides anthocyanin production, also modification and translocation of anthocyanins into vacuoles.

The basic structure of the anthocyanin biosynthetic pathway is conserved across all flowering plants. Nevertheless, characterization of the anthocyanin biosynthetic pathway in other plants, 10) such as petunia (Petunia hybrida), maize (Zea mays), snapdragon (Antirrhinum majus), grapes (Vitis vinifera), and apple (Malus domestica), has shown that species-specific differences exist in this universal metabolic pathway. For example, in maize, all of the biosynthetic genes are regulated by the MBW complex, while in Arabidopsis, the early synthetic genes are regulated by R2R3-MYB proteins. Aside from regulatory differences, changes in the sequences of anthocyanin synthetic genes can alter expression 15 patterns and substrate specificity of the synthetic enzymes. For example, in ornamental flowering plants, such as Senecio cruentus, the enzymes F3′H. F3′5′H, and DER competitively produce cyanidin (Cy), delphinidin (Dp), and pelargonidin (Pg), which are the precursors of red, blue, and orange anthocyanins. Changes in the activity of one of these enzymes cause different combinations of red, blue, and orange anthocyanins to accumulate, which change the flower color. New colours can also be obtained by changing the anthocyanin pattern of methylation, glycosylation, and acylation.

In many plant species, the regulatory and synthetic genes of the anthocyanin pathway have undergone gene duplication events enabling widespread sequence, expression, and regulatory divergence. For example, in strawberries, two DER genes exist, which encode enzymes with different substrate specificities. One variant does not accept flavanonol dihydrokaempferol (DHK) as a substrate, whereas the other strongly prefers DHK. In addition to substrate specificity, the two paralogs also have different expression patterns. Expression of DFR2 is dependent on fruit development, whereas DFR1 expression is relatively stable.

In the species B. oleracea, the number of anthocyanin biosynthetic genes has also expanded through whole genome and tandem duplication during evolution. In Arabidopsis, 41 anthocyanin biosynthetic genes can be identified in the genome, while the genome of B. oleracea contains up to 88 putative anthocyanin biosynthetic genes. The exact number varies between B. oleracea cultivars however. For example, in broccoli not only the late anthocyanin biosynthetic genes, such as DRF and ANS, have duplicated, but the number of R2R3-MYB transcription factors has expanded as well.

In addition to variations in the number of anthocyanin synthetic genes, cultivars also contain different mutations in the promoter and coding sequences of these genes. These mutations lead to significant expression differentiation between paralogs in B. oleracea. As a result, each cultivar contains a unique version of the anthocyanin synthesis pathway with a different set of anthocyanin synthetic and regulatory genes. Due to these differences between cultivars, the mechanisms underlying anthocyanin accumulation in many vegetable crops of B. oleracea are still unknown.

In kohlrabi, cabbage, kale, and cauliflower, purple cultivars are obtained by upregulation of the R2R3-MYB transcription factor, MYB2. MYB2 increases the transcription of anthocyanin biosynthetic genes. In cauliflower, MYB2 is upregulated by the insertion of a transposon, while in cabbage, kale, and kohlrabi MYB2 is upregulated by a point mutation and/or 1-bp insertion in the MYB2 promoter region. Although all four cultivars accumulate anthocyanin in response to MYB2 upregulation, there are striking differences in the distribution of anthocyanin accumulation. While kale accumulates anthocyanins in the leaves and stems, kohlrabi accumulates anthocyanins mainly in the stems and only slightly in the leaves. In cauliflower, anthocyanins accumulate in the curd, but in very few other tissues. In red cabbage, anthocyanins are only found in the epicuticle. The cause of these differences in anthocyanin accumulation between tissues and cultivars is unknown and suggests additional factors control anthocyanin production in different tissues.

Also in broccoli tissue-specific regulation of anthocyanin accumulation can be observed. For example, no purple colour is observed in the leaves of the broccoli line DH16-2 even though its flowerhead is purple and becomes deep purple in cold conditions. A major locus and two minor loci on chromosome C1 have been associated with the purple trait of the flower head in broccoli.

Aside from tissue-specific effects, the production of anthocyanin biosynthesis is also affected by environmental stress. In broccoli, green cultivars can accumulate anthocyanins due to various environmental factors, in particular cold conditions.

As the accumulation of anthocyanins causes a slight purple pigmentation, which reduces the commercial value of the broccoli, the development of anthocyanin-free broccoli is an important target for breeding.

### SUMMARY OF THE INVENTION

It is an object of the present invention, amongst other objects, to meet the above unmet need in the art.

The present invention meets the above object, amongst other objects, as outlined in the appended claims.

Specifically, the above object, amongst other objects, is met by providing an anthocyanin-free broccoli plant comprising a combination of an inactive MYB2 gene and an inactive DFR1 gene. Preferably, the anthocyanin-free broccoli plant comprises both the inactive MYB2 gene and the inactive DFR1 gene homozygously.

The present inventors have surprisingly found that a broccoli plant does not accumulate anthocyanins in response to abiotic stresses such as drought, high light intensity, and, in particular, cold when the broccoli plant comprises a combination of an inactive MYB2 gene and an inactive DFR1 gene.

The inactive MYB2 gene results in an absence or lack of a functional MYB2 protein, whereas the inactive DFR1 gene results in an absence or lack of a functional DFR1 protein. A complete lack or a biologically inadequate amount of MYB2 or DFR1 protein can be caused by various mechanisms, including but not limited to an absence of or a reduced level of MYB2 or DFR1 mRNA. Alternatively, an inactive DFR or MYB2 gene can lead to the production of an inactive MYB2 or DFR1 protein. An inactive MYB2 or DFR1 protein can be, amongst other reasons, non-functional due to a modification in its wild-type amino acid sequence, including deletion and/or insertion of one or more amino acids that destroy the wild-type functionality of the protein. The simultaneous lack of a functional MYB2 protein and DFR1 protein prevents the accumulation of anthocyanins in a broccoli plant.

Preferably, the inactive MYB2 gene is located on chromosome 6 and the inactive DFR1 gene on chromosome 9.

According to a preferred embodiment, the present inactive DFR1 gene is obtained from kale (Brassica oleracea var. acephala). Preferably, the inactive DFR1 gene from kale comprises a 1-bp insertion in the second exon of the DFR1 gene. This nonsense mutation causes premature termination of translation due to a frameshift in the reading frame, which leads to the production of a truncated DFR1 protein that is functionally inactive.

According to another preferred embodiment, the present inactive DFR1 gene is inactivated as a result of a premature termination of transcription and/or translation.

According to yet another preferred embodiment, the present inactive DFR1 gene is inactivated by a mutation. Preferably, the mutation is a mutation in the promotor and/or coding region of the DFR1 gene. More preferably, the mutation is an insertion in and/or a deletion of part or all of the DFR1 gene.

The indefinite article “a” or “an” can be used interchangeably with the phrases “one or more” or “at least one”. As such, the articles “a” and “an” should be understood to mean one, two, three, 30) four, five, six, or more.

The present invention, according to another aspect, relates to seeds or plant parts of an anthocyanin-free broccoli plant. The present seeds are preferably polished, coated, encrusted, pelleted, and/or primed.

The present invention also relates to methods for preventing purple discoloration of a broccoli plant, wherein the method comprises the step of cultivating or growing an anthocyanin-free broccoli plant according to the present invention. The purple discoloration of a broccoli plant, especially the curd, is caused by the accumulation of anthocyanins, which leads to a purple pigmentation.

The present invention also encompasses the use of the combination of an inactive MYB2 and an inactive DFR1 gene to prevent purple pigmentation in a broccoli plant, in particular in the curd of a broccoli plant, preferably wherein the purple pigmentation in a broccoli plant occurs after exposure of the broccoli to cold, and to provide an evergreen broccoli plant. An evergreen broccoli plant is a broccoli plant that does not accumulate purple pigmentation after exposure to abiotic stresses, preferably cold. Preferably, the combination of an inactive MYB2 and an inactive DFR1 gene is used in a homozygous manner to prevent purple pigmentation in a broccoli plant.

The present invention also relates to methods for providing an anthocyanin-free broccoli plant according to the present invention. Such methods preferably comprise the step of inactivating the DFR1 and/or MYB2 gene by introducing a mutation. Mutations can be, but are not limited to, the deletion, substitution, or insertion of nucleic acids in the coding sequence of the DFR1 gene or MYB2 gene.

Mutations can be introduced by mutagenesis or a genome editing technique, preferably CRISPR-Cas. Mutagenesis can be performed using a chemical compound, such as ethyl methane sulphonate (EMS) or N-methyl-N-nitrosourea (MNU), or irradiation techniques, such as UV-irradiation. Examples of genome editing techniques are zinc-finger nucleases (ZFN), transcription activator-like effector nucleases (TALENS), or Clustered Regularly Interspaced Short Palindromic Repeat (CRISPR).

The present invention also provides a method for providing an anthocyanin-free broccoli according to the present invention, wherein the method comprises the steps of crossing a broccoli comprising an inactive MYB2 gene and a kale comprising an inactive DFR1 gene to obtain an F1 plant; backcrossing the F1 plant with a broccoli comprising an inactive MYB2 gene, preferably the broccoli plant used as parent line to generate the F1 plant, to obtain a backcrossed plant; selfing the backcrossed plant to obtain an inbred plant; and selecting an inbred plant comprising a combination of an inactive MYB2 gene and an inactive DFR1 gene.

Backcrossing and selfing are performed at least once. However, the steps of backcrossing and selfing may, independently of each other, be performed any number of times as required or desired to obtain a broccoli line with sufficient commercial quality and/or homozygosity.

The presence of an inactive MYB2 gene and inactive DFR1 gene in a plant is preferably determined after each step of crossing, backcrossing and selfing. The presence of an inactive MYB2 gene and inactive DFR1 gene in a plant can be determined using sequencing methods, such as PCR followed by Sanger sequencing, or marker-assisted selection (MAS), in particular single nucleotide polymorphism (SNP) markers.

### BRIEF DESCRIPTION OF THE DRAWING

The present invention will be further detailed in the examples below. In the examples, reference is made to a figure wherein:

FIG. 1: shows a comparison between the anthocyanin discolored curd of the original parental line A (top) comprising an inactive MYB2 gene and an active DFR1 gene and the two green curds of the NIL line (bottom) comprising an inactive DFR1 gene and inactive MYB2 gene after the parental line and the NIL lines were exposed to cold.

### DESCRIPTION OF THE INVENTION

### EXAMPLES

### Example 1. Development of Anthocyanin-Free Broccoli

For the development of an anthocyanin-free broccoli an introgression library was developed. An introgression library is a plant population build up from a parental line containing small DNA introgressions from another parental line. For this purpose, an F1 cross between broccoli parental line A and kale parental line B was made. The F1 was used for subsequent backcrossing with parental line A and marker assisted selection of specific introgressed DNA fragments. From these backcrossing steps, new homozygous near-isogenic lines (NIL) were developed through inbreeding generations.

The effect of the small introgressions on the phenotype of the lines was compared to parental line A under field conditions. Both line A and line B have a green phenotype, but the resulting F1 cross showed anthocyanin discoloration. This indicated that the “blocked” anthocyanin pathways of both green parental lines were complemented in the resulting F1.

Comparison of the new Brassica introgression library lines showed the involvement of two introgressions situated respectively on chromosome 6 and chromosome 9. The NIL with the combination of the introgression from broccoli on chromosome 6 and the introgression from kale on chromosome 9 resulted in an anthocyanin-free broccoli line under diverse circumstances. Both introgressions were needed to fully block the anthocyanin pathway in broccoli and to obtain an anthocyanin-free broccoli.

### Example 2. Fine Mapping Anthocyanin Pathway and Transcription Factor Genes

The NIL with the small kale introgression on chromosome 6 harbored the MYB2 gene. An additional dedicated backcross population fine-mapped the co-segregation of the purple trait and the MYB2 gene (SEQ ID No. 1; BOLC6T39061H) located on chromosome C6: 37,571,911-37,573,320 of the Brassica oleracea HDEM genome (GenBank: LR031880.1).

The NIL with the kale introgression on chromosome 9 harbored both the TT8 and DFR1 genes involved in the anthocyanin pathway. This specific NIL was used for additional backcrossing and recombination selection. From this backcross population specific plants were selected that harbored a recombination between the TT8 and DFR1 genes. Phenotyping of these recombinants showed co-segregation of the DFR1 gene (SEQ ID No. 2; Bo9g058630.1) with the anthocyanin content. The DFR1 gene is located located on chromosome C9: 17,116,312-17,117,891 of the Brassica oleracea TO1000 genome (GenBank GCA_000695525.1).

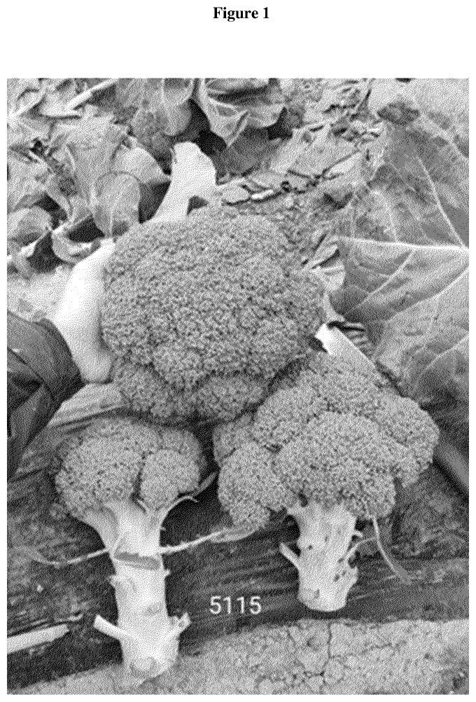

FIG. 1 shows a comparison between the anthocyanin discolored curd of the original parental line A (top) and the two green curds of the NIL line (bottom).

TABLE 1
Sequence information

| SEQ ID No. | Sequence |
|---|---|
| SEQ ID No. 1 | ATGGAGGGTATGTCCAAAGGGTTGAAAAAAGGTGCATGGACTGCTGAAGAAGATAA |
| | TCTCTTGAGGCAATGCATTGATAAGTATGGAGAAGGGAAATGGCACCAAGTTCCTTT |
| | AAGAGCTGGTATGTCTTTTTTTTTGATAAAATAAGAGCTGGTATGCTACTTTTATTAA |
| | TTTTCACACACACACACACACACATATATACATATACATATAACTAATAAGTACGTA |
| | TATTCTTTTTATTTTTCAGTACATTTATTCTCTTTCTCTCTGTCTACTATTAGGAAATTA |
| | ATTAACACCGGGGTACACAATCATTGTTTTTCTTTTCGTTTTAATGAAGGAATCATAG |
| | ATTCAAATGTTCCAATGTTTTTCATGAGAAAAAAAAATATTTGCGTTCTTCATGTTTA |
| | AGTATAAAGCGAGAAGGCAACTCTCTTTATTGATTCGTAGTTTTTTTTGGAGAAATA |
| | GCTTTTTTTTATTTGTGAAATTTTCTGCACGAACCAGTGTGTTTGTGTGGAATATGTTG |
| | TTTATGCTGGTGTACTTTGATTCTTCATGATAAAATTTCAGGAGACGCGAATGCAGTT |
| | TTTGCTCGTTCTTTTAATAATATTAAATGTCAATTGGTTTTGTAGGTCTAAATCGGTG |
| | CAGGAAGAGTTGTAGACTAAGATGGTTGAACTATTTGAAGCCAAGTATCAAGAGAG |
| | GAAAACTCAACTCTGATGAAGTTGATCTTCTTATTCGCCTTCATAAGCTTTTAGGAAA |
| | CAGGTTTACATTCAAGACACAAATTCAACTTTATTTCGTATCCTCATTCGGTCTAATC |
| | TAATCATGTGATTTGTTTTTTTTTGATAAAAAGTACTTAAATTTTTTTCATATGTAAAT |
| | GATCCATTACTAAGTCATATATATCCCTAATTTTTCAAATGCATGCTTAGGTGGTCTT |
| | TAATTGCTGGTAGATTACCCGGTCGGACCGCAAATGACGTCAAAAATTACTGGAACA |
| | CCCATTTGAGTAGGAAACATGAACCAGGTTGTAAGACCCAGATGAAAAAGAGAAAC |
| | ATTCCTTGCTCTTATACCACACCAGCCCAAAAAATCGACGTTTTCAAACCTCGACCTC |
| | GATCCTTCACCGTTAACAACGGCTGCAGCCATATTAATGGCATGGCAGAAGCTGACA |
| | TTGTTCCTCTATGCCTTGGACTCAACGACACTAATAATGTTTCTGAAAATATAATCAC |
| | ATGTAACAAAGATGATGATAAATTTGAGCTTGTTAGTAATTTAATGGATGGTCAGAA |
| | TAGGTGGTGGGAAAGTTTGCTAGATGAGAGCCAAGATCCAGCTGCGCTCTTTCCAGA |
| | AGCTACAGCAACAAAAAAGGGCGCAACCTCCGCGTTTGACGTTGAGCAACTTTGGA |
| | GCCTGTTGGATGGAGAAACTGGAACTTGA |
| SEQ ID No. 2 | ATGGTAGCTCACAAAGAGACCGTGTGCGTAACCGGCGCATCAGGATTCATTGGTTCA |
| | TGGCTCGTGATGCGGCTACTGGAACGTGGTTACTTTGTCCGTGCCACTGTTCGCGATC |
| | CTGGTACGTATCTTACAAACTCGTTAATTTCTCCTAAGAGTATATGTTAATACGTATC |
| | ACTTTGTGTGTTTTAAGTAACTTACGAGTTTTCTTGGCCTGTAAAGGAAATTTGAAGA |
| | AAGTGCAACATCTTCTTGATTTGCCAAACGCGAAGACGCAACTCACTTTATGGAAAG |
| | CCGATTTATCTGACGAAGGAAGCTACGATGACGCCATAAACGGATGCGACGGCGTTT |
| | TTCACATAGCTACTCCCATGGATTTTGAATCCAAGGATCCCGAGGTGAGTTATACTAT |
| | GAACCTTTTTCTTATTACACATCAATCCTACAAGATTTTGTTAAATGAGTTTGTTTGA |
| | ATCAGAACGAAGTGATAAAACCAACAGTGAATGGAGTGTTGGGGATAATGAAAGCA |
| | TGTGATAAGGCAAAGACCGTACGAAGAATTGTGTTTACTTCGTCTGCTGGAACGGTT |
| | AATGTTGAGGAACACCAGAAAAATGTCTATGATGAAAACGATTGGAGTGATCTCGA |
| | CTTTATCATGTCCAAGAAGATGACAGGATGGGTATATATATTAAGGATCATATATAA |
| | AAAATTAACCCGAGGTTGATCTTCTTCAAAGTAATTTATGTTTTTGATAAATTGTTGG |
| | CAGATGTATTTCATGTCGAAAACGTTAGCCGAGAAAGCAGCTTGGGATTACGCTAAG |
| | GAAAAAGGAATAGATTTCATTAGTATTATCCCGACATTGGTGATCGGTCCATTTATA |
| | ACAACATCTATGCCGCCTAGCCTTATTACCGCGCTCTCTCCTATCACTCGTGAGTGAG |
| | CCTACTTTCTAATCCCTCTTTTTTAACTAAGAGGTTAATTTAAAACGGTAAAAATGTT |
| | TTAGGTAACGAGGCACATTACTCCATCATAAGACAAGGACAGTATGTCCACTTGGAC |
| | GACTTATGCAATGCTCATATATTCTTGTACGAACAAGCTGCTGCCAAGGGACGTTAT |
| | GTTTGTTCCTCTCACGATGCAACGATTCTTACTATCTCCGAGTTTCTCAGGCAAAAAT |
| | ATCCAGAATATAACGTGCCTTCAACGTAAGATTTTTATCATTACCGGTTTAAGCTTTT |
| | TTTCCATATTCAGTTTAATTTTTTTTTTTCTGAATATGAACTCTTTGGAACAGGTTTGA |
| | AGGAGTGGATGAGAATCTAAAGAGCATTATGTTCAGTTCCAAGAAGCTGATTGATAT |
| | GGGATTTAACTTCAAGTATAGTCTCGAGGATATGTTGGTGGAATCGATTGAGACATG |
| | TCGTCAAAAGGGTTTTCTCCCTGTCACTTTACCGGAACATTTGAAATCTGAGGACAA |
| | AGTTCCGGGCAGTGATGACAATAAGGAGATTAAAAACGGATCTGCAGGTTTAACTG |
| | ATGGTATGGTAGCTTGTAAGAAGACCGAACCAGGGATGGCCGGCGAGAAAGCCGAT |
| | AGTCACATGTCGGCACAGCAGATCTGTGCTTAG |

### Example 3. CRISPR/Cas9 Knockout of DFR1

To assess if DFR1 is indeed a functional gene for anthocyanin production in broccoli, a knockout of DFR1 was made using CRISPR/Cas9 in a broccoli comprising inactive MYB2 gene. Four target sites were identified (SEQ ID No. 3, 4, 5 and 6) and used to generate guide RNAs (gRNAs). The CRISPR machinery, gRNA and Cas9 proteins, were delivered in broccoli plant cells, via either PEG mediated transfection of ribonucleoproteins (RNP), or Agrobacterium-mediated gene transfer. Edited cells were regenerated to obtain viable plants. Broccoli plants without an active DFR1 gene showed the expected phenotype.

TABLE 2
Identified target sites for generating
CRISPR/Cas9 knockouts of DFR1

| SEQ ID No. | Sequence |
|---|---|
| SEQ ID No. 3 | CATCAGGATTCATTGGTTCATGG |
| SEQ ID No. 4 | GTGAGTTGCGTCTTCGCGTTTGG |
| SEQ ID No. 5 | GATAATGAAAGCATGTGATAAGG |
| SEQ ID No. 6 | AGCAGCTTGGGATTACGCTAAGG |

### Example 4. Identification of a DFR1 Mutant from a TILLING Population

To assess if DFR1 is indeed a functional gene for anthocyanin production in broccoli, a DFR1 mutant allele can be found in a broccoli TILLING population by screening and/or resequencing.

The TILLING population was generated by treating seeds of a broccoli with an inactive MYB2 gene with Ethyl Methyl Sulfate (EMS). Surviving seedlings were grown into plants and subsequently selfed to obtain M2 seeds. The M2 seeds can be sampled to identify DFR1 mutants. Plants that have a nonsense mutation in DFR1 can be selected and backcrossed several times, while selecting for the mutation. This will result in an anthocyanin-free broccoli plant due to the absence of active MYB2 and DFR1 genes.

1. An anthocyanin-free broccoli plant comprising a combination of an inactive MYB2 gene and an inactive DFR1 gene.

2. The anthocyanin-free broccoli plant according to claim 1, wherein the inactive MYB2 gene is located on chromosome 6 and the inactive DFR1 gene is located on chromosome 9.

3. The anthocyanin-free broccoli plant according to claim 1, wherein the inactive DFR1 gene is obtained from kale.

4. The anthocyanin-free broccoli plant according to claim 1, wherein the inactive DFR1 gene is inactive as a result of a premature termination of transcription and/or translation.

5. The anthocyanin-free broccoli plant according to claim 1, wherein the inactive DFR1 gene comprises a mutation, wherein the mutation results in an absence of a functional DFR1 protein.

6. The anthocyanin-free broccoli plant claim 5, wherein the mutation is an insertion in and/or a deletion of part or all of the DFR1 gene.

7. The anthocyanin-free broccoli plant according to claim 1, wherein the inactive DFR1 gene encodes an inactive DFR1 protein.

8. Seeds or plant parts of an anthocyanin-free broccoli plant according to claim 1.

9. A method for preventing purple discoloration of a broccoli plant, wherein the method comprises the step of cultivating or growing an anthocyanin-free broccoli plant according to claim 1.

10-12. (canceled)

13. A method for providing an anthocyanin-free broccoli plant comprising inactivating the DFR1 and/or the MYB2 gene by introducing a mutation.

14. The method according to claim 13, wherein the mutation is introduced by mutagenesis or a genome editing technique.

15. A method for providing an anthocyanin-free broccoli plant, wherein the method comprises the steps of:
a) crossing a broccoli plant comprising an inactive MYB2 gene and a kale plant comprising an inactive DFR1 gene to obtain an F1 plant;
b) backcrossing the F1 plant with the broccoli plant comprising an inactive MYB2gene to obtain a backcrossed plant;
c) selfing a backcrossed plant to obtain an inbred plant; and
d) selecting an inbred plant comprising a combination of an inactive MYB2 gene and an inactive DFR1 gene.


SEQUENCE LISTING (6 sequences)

SEQ ID NO: 1 - DNA, 1410 bp, Brassica oleracea
  source 1..1410: mol_type = genomic DNA; strain = var. italica
atggagggtatgtccaaagggttgaaaaaaggtgcatggactgctgaagaagataatctcttgaggcaatgcattgataagtatggagaagggaaatggcaccaagttcctttaagagctggtatgtctttttttttgataaaataagagctggtatgctacttttattaattttcacacacacacacacacacatatatacatatacatataactaataagtacgtatattctttttatttttcagtacatttattctctttctctctgtctactattaggaaattaattaacaccggggtacacaatcattgtttttcttttcgttttaatgaaggaatcatagattcaaatgttccaatgtttttcatgagaaaaaaaaatatttgcgttcttcatgtttaagtataaagcgagaaggcaactctctttattgattcgtagttttttttggagaaatagcttttttttatttgtgaaattttctgcacgaaccagtgtgtttgtgtggaatatgttgtttatgctggtgtactttgattcttcatgataaaatttcaggagacgcgaatgcagtttttgctcgttcttttaataatattaaatgtcaattggttttgtaggtctaaatcggtgcaggaagagttgtagactaagatggttgaactatttgaagccaagtatcaagagaggaaaactcaactctgatgaagttgatcttcttattcgccttcataagcttttaggaaacaggtttacattcaagacacaaattcaactttatttcgtatcctcattcggtctaatctaatcatgtgatttgtttttttttgataaaaagtacttaaatttttttcatatgtaaatgatccattactaagtcatatatatccctaatttttcaaatgcatgcttaggtggtctttaattgctggtagattacccggtcggaccgcaaatgacgtcaaaaattactggaacacccatttgagtaggaaacatgaaccaggttgtaagacccagatgaaaaagagaaacattccttgctcttataccacaccagcccaaaaaatcgacgttttcaaacctcgacctcgatccttcaccgttaacaacggctgcagccatattaatggcatggcagaagctgacattgttcctctatgccttggactcaacgacactaataatgtttctgaaaatataatcacatgtaacaaagatgatgataaatttgagcttgttagtaatttaatggatggtcagaataggtggtgggaaagtttgctagatgagagccaagatccagctgcgctctttccagaagctacagcaacaaaaaagggcgcaacctccgcgtttgacgttgagcaactttggagcctgttggatggagaaactggaacttga

SEQ ID NO: 2 - DNA, 1580 bp, Brassica oleracea
  source 1..1580: mol_type = genomic DNA; strain = var. acephala
atggtagctcacaaagagaccgtgtgcgtaaccggcgcatcaggattcattggttcatggctcgtgatgcggctactggaacgtggttactttgtccgtgccactgttcgcgatcctggtacgtatcttacaaactcgttaatttctcctaagagtatatgttaatacgtatcactttgtgtgttttaagtaacttacgagttttcttggcctgtaaaggaaatttgaagaaagtgcaacatcttcttgatttgccaaacgcgaagacgcaactcactttatggaaagccgatttatctgacgaaggaagctacgatgacgccataaacggatgcgacggcgtttttcacatagctactcccatggattttgaatccaaggatcccgaggtgagttatactatgaacctttttcttattacacatcaatcctacaagattttgttaaatgagtttgtttgaatcagaacgaagtgataaaaccaacagtgaatggagtgttggggataatgaaagcatgtgataaggcaaagaccgtacgaagaattgtgtttacttcgtctgctggaacggttaatgttgaggaacaccagaaaaatgtctatgatgaaaacgattggagtgatctcgactttatcatgtccaagaagatgacaggatgggtatatatattaaggatcatatataaaaaattaacccgaggttgatcttcttcaaagtaatttatgtttttgataaattgttggcagatgtatttcatgtcgaaaacgttagccgagaaagcagcttgggattacgctaaggaaaaaggaatagatttcattagtattatcccgacattggtgatcggtccatttataacaacatctatgccgcctagccttattaccgcgctctctcctatcactcgtgagtgagcctactttctaatccctcttttttaactaagaggttaatttaaaacggtaaaaatgttttaggtaacgaggcacattactccatcataagacaaggacagtatgtccacttggacgacttatgcaatgctcatatattcttgtacgaacaagctgctgccaagggacgttatgtttgttcctctcacgatgcaacgattcttactatctccgagtttctcaggcaaaaatatccagaatataacgtgccttcaacgtaagatttttatcattaccggtttaagctttttttccatattcagtttaatttttttttttctgaatatgaactctttggaacaggtttgaaggagtggatgagaatctaaagagcattatgttcagttccaagaagctgattgatatgggatttaacttcaagtatagtctcgaggatatgttggtggaatcgattgagacatgtcgtcaaaagggttttctccctgtcactttaccggaacatttgaaatctgaggacaaagttccgggcagtgatgacaataaggagattaaaaacggatctgcaggtttaactgatggtatggtagcttgtaagaagaccgaaccagggatggccggcgagaaagccgatagtcacatgtcggcacagcagatctgtgcttag

SEQ ID NO: 3 - DNA, 23 bp, Brassica oleracea
  source 1..23: mol_type = other DNA; strain = var. italica
catcaggattcattggttcatgg

SEQ ID NO: 4 - DNA, 23 bp, Brassica oleracea
  source 1..23: mol_type = other DNA; strain = var. italica
gtgagttgcgtcttcgcgtttgg

SEQ ID NO: 5 - DNA, 23 bp, Brassica oleracea
  source 1..23: mol_type = other DNA; strain = var. italica
gataatgaaagcatgtgataagg

SEQ ID NO: 6 - DNA, 23 bp, Brassica oleracea
  source 1..23: mol_type = other DNA; strain = var. italica
agcagcttgggattacgctaagg